(19) United States
(12) Patent Application Publication
(10) Pub. No.: US 2023/0345222 A1
(43) Pub. Date: Oct. 26, 2023

(54) Emergency Alert Watch Assembly

(71) Applicant: Idell Clark, Oceanside, CA (US)

(72) Inventor: Idell Clark, Oceanside, CA (US)

(21) Appl. No.: 17/725,301

(22) Filed: Apr. 20, 2022

(51) Int. Cl. H04W 4/90; H04N 7/18; G06V 40/13; G06V 40/12; A61B 5/0205; A61B 5/024; A61B 5/00; G04G 21/04; G04G 21/02

(52) U.S. Cl. CPC H04W 4/90; H04N 7/185; G06V 40/13; G06V 40/1365; A61B 5/02055; A61B 5/02438; A61B 5/681; A61B 5/7425; A61B 5/747; G04G 21/04; G04G 21/025

(57) ABSTRACT

An emergency alert watch assembly includes a watch is wearable on a wrist of a user. A display is integrated into the watch to displaying indicia comprising numbers indicating a time of day. A communication unit is integrated into the watch. The communication unit is in remote communication with an extrinsic communication network thereby facilitating the communication unit to be in communication with emergency responders. The communication unit broadcasts an assistance request to the emergency responders when the communication unit is actuated. A fingerprint scanner is integrated into the watch to read a fingerprint of the user. A camera is integrated into the watch facilitating the emergency responders to view the user when the communication unit is actuated.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to watch devices and more particularly pertains to a new watch device for facilitating a user to request emergency assistance from emergency responders. The device includes a wrist watch and a communication unit integrated into the wrist watch. The communication unit broadcasts a request for assistance to emergency responders when the communication unit is actuated. The device includes a fingerprint scanner integrated into the watch to can the user's fingerprint. The device includes a speaker and a microphone to facilitate the user to communicate with the emergency responders.

### (2) Description of Related Art Including Information Disclosed Under 37 CFR 1.97 and 1.98

The prior art relates to watch devices including a child monitoring system that includes a bracelet worn on a child, a camera device worn on a care giver's clothing and a receiver worn on the care giver's clothing. The prior art discloses a variety of child tracking units that includes a tracking module worn on a child and a control unit in possession of a caregiver. The prior art discloses a personal alarm watch device that includes a pulse monitor, a global positioning tracker and a transceiver in communication with an analysis array. The prior art discloses a wrist worn monitoring device that includes an accelerometer, an altimeter and a thermometer for transmitting data to a health care provider. The prior art discloses a health monitoring device that includes a bracelet, a plurality of physiological sensors for sensing a physiological condition of a user and audio streaming capabilities.

### BRIEF SUMMARY OF THE INVENTION

An embodiment of the disclosure meets the needs presented above by generally comprising a watch is wearable on a wrist of a user. A display is integrated into the watch to displaying indicia comprising numbers indicating a time of day. a communication unit is integrated into the watch. The communication unit is in remote communication with an extrinsic communication network thereby facilitating the communication unit to be in communication with emergency responders. The communication unit broadcasts an assistance request to the emergency responders when the communication unit is actuated. A fingerprint scanner is integrated into the watch to read a fingerprint of the user. A camera is integrated into the watch facilitating the emergency responders to view the user when the communication unit is actuated.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top phantom view of an emergency alert watch assembly according to an embodiment of the disclosure.

FIG. 2 is a bottom phantom view of an embodiment of the disclosure.

FIG. 3 is a front view of an embodiment of the disclosure.

FIG. 4 is a right side view of an embodiment of the disclosure.

FIG. 5 is a cross sectional view taken along line 5-5 of FIG. 4 of an embodiment of the disclosure.

FIG. 6 is a perspective in-use view of an embodiment of the disclosure.

### DETAILED DESCRIPTION OF THE INVENTION

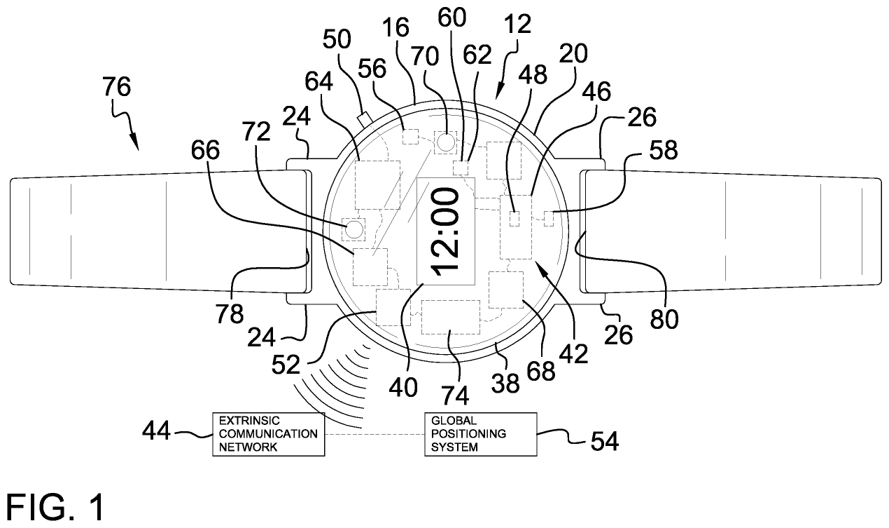

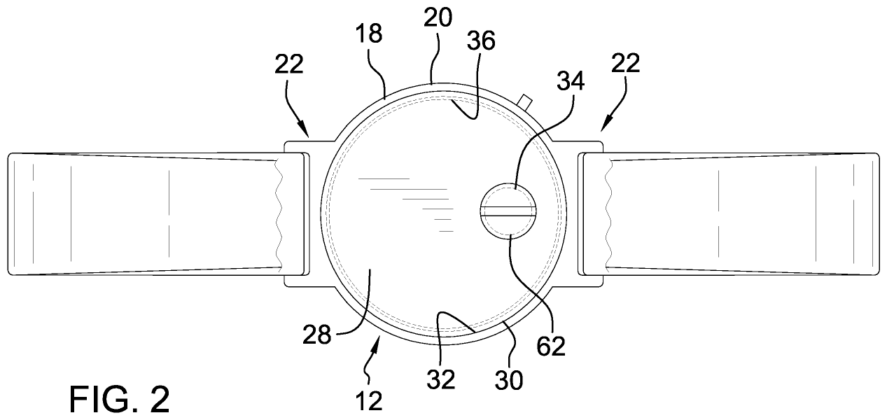

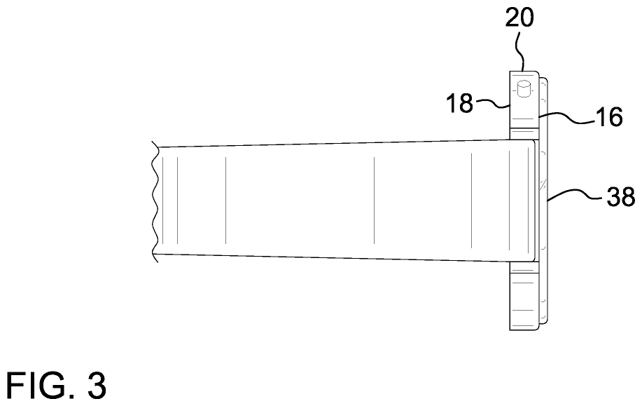

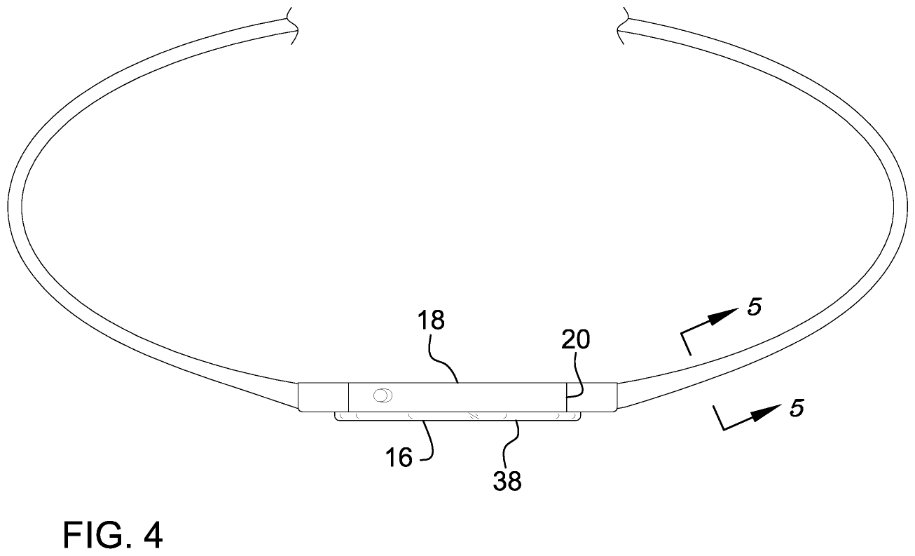

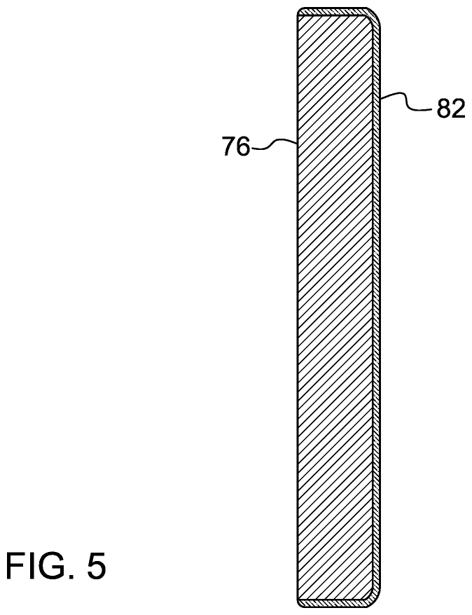

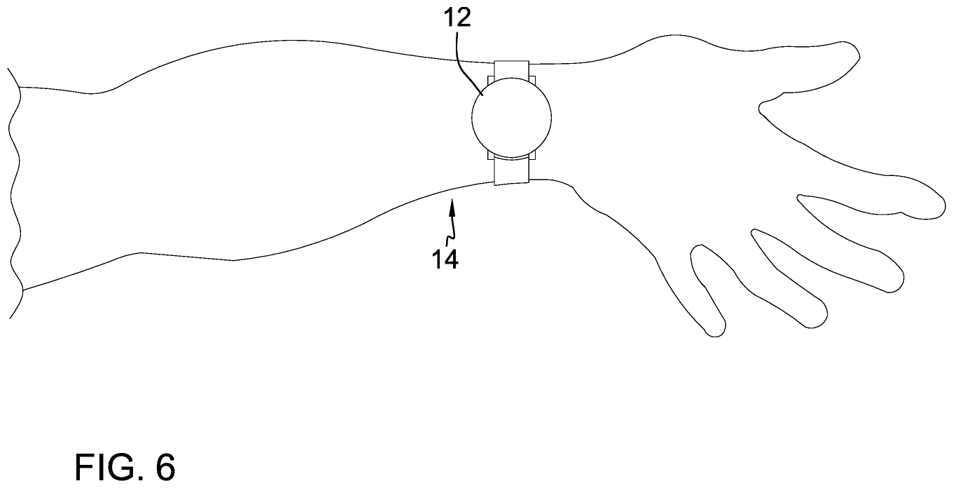

With reference now to the drawings, and in particular to FIGS. 1 through 6 thereof, a new watch device embodying the principles and concepts of an embodiment of the disclosure and generally designated by the reference numeral 10 will be described.

As best illustrated in FIGS. 1 through 6, the emergency alert watch assembly 10 generally comprises a watch 12 that is wearable on a wrist 14 of a user. The watch 12 has a top side 16, a bottom side 18 and a perimeter surface 20 extending between the top side 16 and the bottom side 18, and the watch 12 has a plurality of mounting tabs 22 each extending away from the perimeter surface 20. The plurality of mounting tabs 22 includes a set of first mounting tabs 24 and a set of second mounting tabs 26. The first mounting tabs 24 are positioned on an opposing side of the perimeter surface 20 from the second mounting tabs 26. Furthermore, the first mounting tabs 24 are spaced apart from each other and the second mounting tabs 26 are spaced apart from each other.

The bottom side 18 of the watch 12 is open and a cover 28 is removably coupled to the bottom side 18 of the watch 12 for closing the bottom side 18. The cover 28 has a perimeter edge 30 and the perimeter edge 30 is coextensive with a bounding edge 32 of the bottom side 18 of the watch 12. Additionally, the cover 28 has a battery cap 34 that is removably integrated into the cover 28. A gasket 36 is positioned between the cover 28 and the watch 12. The gasket 36 is comprised of a resiliently compressible material to form a fluid impermeable seal with the watch 12. In this way the gasket 36 inhibits fluid, such as water, from entering into an interior of the watch 12. A lens 38 is integrated into the top side 16 of the watch 12 and the lens 38 is comprised of a translucent material.

A display 40 is integrated into the watch 12 and the display 40 displays indicia comprising numbers indicating a time of day. The display 40 is positioned on the top side 16 of the watch 12 and the display 40 may comprise a liquid crystal display or other type of electronic display. A communication unit 42 is integrated into the watch 12 and the communication unit 42 is in remote communication with an extrinsic communication network 44 thereby facilitating the communication unit 42 to be in communication with emergency responders. Furthermore, the communication unit 42 broadcasts an assistance request to the emergency responders when the communication unit 42 is actuated. The extrinsic communication network 44 may be a cellular phone network or the like and the emergency responders may be a local police department.

The communication unit 42 comprises a control circuit 46 that is integrated into the watch 12. The control circuit 46 receives an alert input and the control circuit 46 is electrically coupled to the display 40. Additionally, the control circuit 46 includes an electronic memory 48 which stores a fingerprint of an authorized user, and the authorized user may be the user upon which the watch 12 is being worn. A control button 50 is movably integrated into the perimeter surface 20 of the watch 12 and the control button 50 is electrically coupled to the control circuit 46. The control circuit 46 receives the alert input when the control button 50 is depressed.

The communication unit 42 includes a transceiver 52 that is integrated into the watch 12, the transceiver 52 is electrically coupled to the control circuit 46 and the transceiver 52 is in wireless communication with the extrinsic communication network 44. The transceiver 52 broadcasts the assistance request to the emergency responders when the control circuit 46 receives the alert input. Additionally, the transceiver 52 is in continuous communication with the emergency responders when the transceiver 52 broadcasts the assistance request. The transceiver 52 is in wireless communication with a global positioning system 54 such that the transceiver 52 receives the physical location of the watch 12 from the global positioning system 54. Moreover, the transceiver 52 broadcasts the physical location to the emergency responders to facilitate the emergency responders to track the location of the user. The transceiver 52 may comprise a radio frequency transceiver or the like and the transceiver 52 may employ a Global System for Mobile Communication signal or other similar protocol thereby facilitating the transceiver 52 to communicate with a cellular phone network. The transceiver 52 may additionally be in communication with a sexual predator database which stores the location of registered sexual predators in the area in which the user is located. In this way the display 40 can potentially display a graphical map which displays the location of the registered sexual predators in and around the location in which the user is currently located.

The communication unit 42 includes a speaker 56 is integrated into the watch 12 to emit audible sounds outwardly from the watch 12. The speaker 56 is electrically coupled to the control circuit 46 thereby facilitating the speaker 56 to receive communication from the emergency responders to facilitate the user to hear spoken words from the emergency responders. Furthermore, the speaker 56 is turned on when the control circuit 46 receives the alert input. The communication unit 42 includes a microphone 58 is integrated into the watch 12 to detect audible sounds. The microphone 58 is electrically coupled to the control circuit 46 thereby facilitating the microphone 58 to transmit communication from the user wherein the microphone 58 is configured to facilitate the emergency responders to hear spoken words from the user. The communication unit 42 includes a power supply 60 that is integrated into the watch 12, the power supply 60 is electrically coupled to the control circuit 46 and the power supply 60 comprises at least one battery 62. Additionally, the power supply 60 is positioned behind the battery cap 34 in the cover 28 for removing and replacing the at least one battery 62.

A fingerprint scanner 64 is integrated into the watch 12 to read a fingerprint of the user. The communication unit 42 is actuated when the fingerprint scanner 64 reads a fingerprint which matches a pre-determined fingerprint. Additionally, the fingerprint scanner 64 is integrated into the top side 16 of the watch 12 and the fingerprint scanner 64 is electrically coupled to the control circuit 46. The control circuit 46 is actuated into a standby mode for receiving the actuate input when the fingerprint scanner 64 scans a fingerprint matching the fingerprint of the authorized user stored in the electronic memory 48. The fingerprint scanner 64 may be an electronic fingerprint scanner of any conventional design. Furthermore, the user's fingerprint is scanned into the electronic memory 48 upon initialization of the communication unit 42 to identify the user's fingerprint as the authorized user's fingerprint.

A heart rate sensor 66 is integrated into the watch 12 and the heart rate sensor 66 is in physical contact with the user when the user wears the watch 12. In this way the heart rate sensor 66 can sense the user's heart rate. The heart rate sensor 66 is electrically coupled to the control circuit 46 and the display 40 displays indicia comprising numerals corresponding to the user's heart rate. The heart rate sensor 66 may comprise an electronic heart rate sensor of any conventional design. A temperature sensor 68 is integrated into the watch 12 and the temperature sensor 68 is in physical contact with the user when the user wears the watch 12 to sense the user's body temperature. Additionally, the temperature sensor 68 is electrically coupled to the control circuit 46 and the display 40 displays indicia comprising numerals corresponding to the user's body temperature. The temperature sensor 68 may comprise an electronic thermometer of any conventional design.

A camera 70 is integrated into the watch 12 to capture imagery of the user. The camera 70 is in communication with the communication unit 42 thereby facilitating the emergency responders to view the user when the communication unit 42 is actuated. The camera 70 is integrated into the top side 16 of the watch 12, the camera 70 is electrically coupled to the control circuit 46 and the camera 70 is turned on when the control circuit 46 receives the alert input. Additionally, the camera 70 may comprise a digital video camera of any conventional design.

A light emitter 72 is integrated into the watch 12 to emit light outwardly from the camera 70 when the light emitter 72 is turned on. The light emitter 72 is in communication with the communication unit 42 and the light emitter 72 is turned on when the communication unit 42 is actuated. In this way the light emitter 72 illuminates the user to facilitate the camera 70 to capture clear imagery of the user. The light emitter 72 is electrically coupled to the control circuit 46 and the light emitter 72 is turned on when the control circuit 46 receives the alert input. The light emitter 72 may comprise a light emitting diode or other type of miniaturized light emitter.

A compass 74 is integrated into the watch 12 thereby facilitating the compass 74 to detect magnetic north. The compass 74 is electrically coupled to the control circuit 46 and the display 40 displays indicia comprising letters for communicating which direction the watch 12 is facing. The compass 74 may be a digital compass of any conventional design. A wrist band 76 is coupled to the watch 12 thereby facilitating the wrist band 76 to extend around the user's wrist 14. The wrist band 76 is comprised of a resilient material to resist being broken. In this way the watch 12 from is inhibited from being forcibly removed from the user's wrist 14.

The wrist band 76 has a first end 78 and a second end 80, and the first end 78 is positioned between each of the first mounting tabs 24 on the perimeter surface 20 of the watch 12. The first end 78 movably engages each of the first mounting tabs 24 and the second end 80 is positioned between each of the second mounting tabs 26 on the perimeter surface 20 of the watch 12. The second end 80 movably engages each of the second mounting tabs 26. The resilient material of which the wrist band 76 is comprised may be stainless steel or other similar metallic alloy that is corrosion resistant and known to not cause allergic skin reactions. A covering 82 is wrapped around the wrist band 76 to enhance the ornamental appearance of the wrist band 76. Furthermore, the covering 82 is comprised of a deformable material, including but not being limited to cow leather, to enhance comfort of the wrist band 76 on the user's wrist 14.

In use, the user scans their fingerprint into the electronic memory 48 upon initialization of the watch 12 and the user's personal information is downloaded into the electronic memory 48 via the transceiver 52. The control button 50 is depressed when the user experiences an emergency. The transceiver 52 contacts the emergency responders, the camera 70 turns on, the speaker 56 turns on and the microphone 58 turns on. In this way the user can communicate with the emergency responders in order to immediately receive assistance. Furthermore, the heart rate sensor 66 and the temperature sensor 68 facilitate the emergency responders to monitor the physical condition of the user. The transceiver 52 broadcasts the physical location of the watch 12 to the emergency responders thereby facilitating the emergency responders to effectively respond to the user's emergency.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An emergency alert watch assembly for facilitating a user to contact emergency responders in the event of a personal emergency, said assembly comprising:
a watch being wearable on a wrist of a user;
a display being integrated into said watch, said display displaying indicia comprising numbers indicating a time of day;
a communication unit being integrated into said watch, said communication unit being in remote communication with an extrinsic communication network thereby facilitating said communication unit to be in communication with emergency responders, said communication unit broadcasting an assistance request to the emergency responders when said communication unit is actuated;
a fingerprint scanner being integrated into said watch wherein said fingerprint scanner is configured to read a fingerprint of the user, said communication unit being actuated when said fingerprint scanner reads a fingerprint which matches a pre-determined fingerprint;
a heart rate sensor being integrated into said watch, said heart rate sensor being in physical contact with the user when the user wears said watch wherein said heart rate sensor is configured to sense the user's heart rate;
a temperature sensor being integrated into said watch, said temperature sensor being in physical contact with the user when said user wears said watch wherein said temperature sensor is configured to sense the user's body temperature;
a camera being integrated into said watch wherein said camera is configured to capture imagery of the user, said camera being in communication with said communication unit thereby facilitating the emergency responders to view the user when said communication unit is actuated;
a light emitter being integrated into said watch wherein said light emitter is configured to emit light outwardly from said camera when said light emitter is turned on, said light emitter being in communication with said communication unit, said light emitter being turned on when said communication unit is actuated wherein said light emitter is configured to illuminate the user to facilitate said camera to capture clear imagery of the user; and
a wrist band being coupled to said watch thereby facilitating said wrist band to extend around the user's wrist, said wrist band being comprised of a resilient material to resist being broken wherein said wrist band is configured to inhibit said watch from being forcibly removed from the user's wrist.

2. The assembly according to claim 1, wherein:
said watch having a top side, a bottom side and a perimeter surface extending between said top side and said bottom side, said watch having a plurality of mounting tabs each extending away from said perimeter surface, said plurality of mounting tabs including a set of first mounting tabs and a second of second mounting tabs, said first mounting tabs being positioned on an opposing side of said perimeter surface from said second mounting tabs, said first mounting tabs being spaced apart from each other, said second mounting tabs being spaced apart from each other, said bottom side of said watch being open;
a cover being removably coupled to said bottom side of said watch for closing said bottom side, said cover having a perimeter edge, said perimeter edge being coextensive with a bounding edge of said bottom side of said watch, said cover having a battery cap being removably integrated into said cover; and
a gasket being positioned between said cover and said watch, said gasket being comprised of a resiliently compressible material wherein said gasket is configured to form a fluid impermeable seal with said watch.

3. The assembly according to claim 1, wherein said communication unit comprises:
a control circuit being integrated into said watch, said control circuit receiving an alert input, said control circuit being electrically coupled to said display, said control circuit including an electronic memory storing a fingerprint of an authorized user;
a control button being movably integrated into said perimeter surface of said watch, said control button being electrically coupled to said control circuit, said control circuit receiving said alert input when said control button is depressed;
a transceiver being integrated into said watch, said transceiver being electrically coupled to said control circuit, said transceiver being in wireless communication with the extrinsic communication network, said transceiver broadcasting said assistance request to the emergency responders when said control circuit receives said alert input, said transceiver being in continuous communication with the emergency responders when said transceiver broadcasts said assistance request; and
said transceiver is in wireless communication with a global positioning system wherein said transceiver is configured to receive the physical location of said watch from the global positioning system, said transceiver broadcasting the physical location to the emergency responders wherein said transceiver is configured to facilitate the emergency responders to track the location of the user.

4. The assembly according to claim 3, wherein said communication unit comprises:
a speaker being integrated into said watch wherein said speaker is configured to emit audible sounds outwardly from said watch, said speaker being electrically coupled to said control circuit thereby facilitating said speaker to receive communication from the emergency responders wherein said speaker is configured to facilitate the user to hear spoken words from the emergency responders, said speaker being turned on when said control circuit receives said alert input; and
a microphone being integrated into said watch wherein said microphone is configured to detect audible sounds, said microphone being electrically coupled to said control circuit thereby facilitating said microphone to transmit communication from the user wherein said microphone is configured to facilitate the emergency responders to hear spoken words from the user.

5. The assembly according to claim 3, wherein said fingerprint scanner is electrically coupled to said control circuit, said control circuit being actuated into a standby mode for receiving said actuate input when said fingerprint scanner scans a fingerprint matching said fingerprint of the authorized user stored in said electronic memory, said fingerprint scanner being integrated into said top side of said watch.

6. The assembly according to claim 3, wherein:
said heart rate scanner is electrically coupled to said control circuit, said display displaying indicia comprising numerals corresponding to the user's heart rate; and
said temperature sensor is electrically coupled to said control circuit, said display displaying indicia comprising numerals corresponding to the user's body temperature.

7. The assembly according to claim 3, wherein:
said camera being integrated into said top side of said watch, said camera being electrically coupled to said control circuit, said camera being turned on when said control circuit receives said alert input; and
said light emitter is electrically coupled to said control circuit, said light emitter being turned on when said control circuit receives said alert input.

8. The assembly according to claim 3, further comprising a compass being integrated into said watch thereby facilitating said compass to detect magnetic north, said compass being electrically coupled to said control circuit, said display displaying indicia comprising letters for communicating which direction said watch is facing.

9. The assembly according to claim 2, wherein:
said wrist band has a first end and a second end, said first end being positioned between each of said first mounting tabs on said perimeter surface of said watch, said first end movably engaging each of said first mounting tabs, said second end being positioned between each of said second mounting tabs on said perimeter surface of said watch, said second end movably engaging each of said second mounting tabs; and
said assembly includes a covering being wrapped around said wrist band wherein said covering is configured to enhance the ornamental appearance of said wrist band, said covering being comprised of a deformable material wherein said covering is configured to enhance comfort of said wrist band on the user's wrist.

10. An emergency alert watch assembly for facilitating a user to contact emergency responders in the event of a personal emergency, said assembly comprising:
a watch being wearable on a wrist of a user, said watch having a top side, a bottom side and a perimeter surface extending between said top side and said bottom side, said watch having a plurality of mounting tabs each extending away from said perimeter surface, said plurality of mounting tabs including a set of first mounting tabs and a second of second mounting tabs, said first mounting tabs being positioned on an opposing side of said perimeter surface from said second mounting tabs, said first mounting tabs being spaced apart from each other, said second mounting tabs being spaced apart from each other, said bottom side of said watch being open;
a cover being removably coupled to said bottom side of said watch for closing said bottom side, said cover having a perimeter edge, said perimeter edge being coextensive with a bounding edge of said bottom side of said watch, said cover having a battery cap being removably integrated into said cover;
a gasket being positioned between said cover and said watch, said gasket being comprised of a resiliently compressible material wherein said gasket is configured to form a fluid impermeable seal with said watch;
a display being integrated into said watch, said display displaying indicia comprising numbers indicating a time of day, said display being positioned on said top side of said watch;
a communication unit being integrated into said watch, said communication unit being in remote communication with an extrinsic communication network thereby facilitating said communication unit to be in communication with emergency responders, said communication unit broadcasting an assistance request to the emergency responders when said communication unit is actuated, said communication unit comprising:
a control circuit being integrated into said watch, said control circuit receiving an alert input, said control circuit being electrically coupled to said display, said control circuit including an electronic memory storing a fingerprint of an authorized user;
a control button being movably integrated into said perimeter surface of said watch, said control button being electrically coupled to said control circuit, said control circuit receiving said alert input when said control button is depressed;
a transceiver being integrated into said watch, said transceiver being electrically coupled to said control circuit, said transceiver being in wireless communication with the extrinsic communication network, said transceiver broadcasting said assistance request to the emergency responders when said control circuit receives said alert input, said transceiver being in continuous communication with the emergency responders when said transceiver broadcasts said assistance request, said transceiver being in wireless communication with a global positioning system wherein said transceiver is configured to receive the physical location of said watch from the global positioning system, said transceiver broadcasting the physical location to the emergency responders wherein said transceiver is configured to facilitate the emergency responders to track the location of the user;
a speaker being integrated into said watch wherein said speaker is configured to emit audible sounds outwardly from said watch, said speaker being electrically coupled to said control circuit thereby facilitating said speaker to receive communication from the emergency responders wherein said speaker is configured to facilitate the user to hear spoken words from the emergency responders, said speaker being turned on when said control circuit receives said alert input;
a microphone being integrated into said watch wherein said microphone is configured to detect audible sounds, said microphone being electrically coupled to said control circuit thereby facilitating said microphone to transmit communication from the user wherein said microphone is configured to facilitate the emergency responders to hear spoken words from the user; and
a power supply being integrated into said watch, said power supply being electrically coupled to said control circuit, said power supply comprising at least one battery;
a fingerprint scanner being integrated into said watch wherein said fingerprint scanner is configured to read a fingerprint of the user, said communication unit being actuated when said fingerprint scanner reads a fingerprint which matches a pre-determined fingerprint, said fingerprint scanner being integrated into said top side of said watch, said fingerprint scanner being electrically coupled to said control circuit, said control circuit being actuated into a standby mode for receiving said actuate input when said fingerprint scanner scans a fingerprint matching said fingerprint of the authorized user stored in said electronic memory;
a heart rate sensor being integrated into said watch, said heart rate sensor being in physical contact with the user when the user wears said watch wherein said heart rate sensor is configured to sense the user's heart rate, said heart rate scanner being electrically coupled to said control circuit, said display displaying indicia comprising numerals corresponding to the user's heart rate;
a temperature sensor being integrated into said watch, said temperature sensor being in physical contact with the user when said user wears said watch wherein said temperature sensor is configured to sense the user's body temperature, said temperature sensor being electrically coupled to said control circuit, said display displaying indicia comprising numerals corresponding to the user's body temperature;
a camera being integrated into said watch wherein said camera is configured to capture imagery of the user, said camera being in communication with said communication unit thereby facilitating the emergency responders to view the user when said communication unit is actuated, said camera being integrated into said top side of said watch, said camera being electrically coupled to said control circuit, said camera being turned on when said control circuit receives said alert input;
a light emitter being integrated into said watch wherein said light emitter is configured to emit light outwardly from said camera when said light emitter is turned on, said light emitter being in communication with said communication unit, said light emitter being turned on when said communication unit is actuated wherein said light emitter is configured to illuminate the user to facilitate said camera to capture clear imagery of the user, said light emitter being electrically coupled to said control circuit, said light emitter being turned on when said control circuit receives said alert input;
a compass being integrated into said watch thereby facilitating said compass to detect magnetic north, said compass being electrically coupled to said control circuit, said display displaying indicia comprising letters for communicating which direction said watch is facing;
a wrist band being coupled to said watch thereby facilitating said wrist band to extend around the user's wrist, said wrist band being comprised of a resilient material to resist being broken wherein said wrist band is configured to inhibit said watch from being forcibly removed from the user's wrist, said wrist band having a first end and a second end, said first end being positioned between each of said first mounting tabs on said perimeter surface of said watch, said first end movably engaging each of said first mounting tabs, said second end being positioned between each of said second mounting tabs on said perimeter surface of said watch, said second end movably engaging each of said second mounting tabs; and
a covering being wrapped around said wrist band wherein said covering is configured to enhance the ornamental appearance of said wrist band, said covering being comprised of a deformable material wherein said covering is configured to enhance comfort of said wrist band on the user's wrist.
